(19) United States
(12) Patent Application Publication
(10) Pub. No.: US 2024/0418587 A1
(43) Pub. Date: Dec. 19, 2024

(54) A SENSOR

(71) Applicant: VEINSENSE LTD, Wadhurst (GB)

(72) Inventors: Arnold HARPIN, Wadhurst (GB); Stephen BARKER, Shirley (GB)

(21) Appl. No.: 18/728,345

(22) PCT Filed: Jan. 9, 2023
(86) PCT No.: PCT/GB2023/050027

§ 371 Date: Jul. 11, 2024

(30) Foreign Application Priority Data
Jan. 12, 2022 (GB) 2200359.4

(51) Int. Cl. G01L 5/00; A61B 5/00; A61B 5/103; A61F 13/00; G01L 1/26

(52) U.S. Cl. CPC G01L 5/0028; A61B 5/1036; A61B 5/6804; A61F 13/00051; G01L 1/26; A61F 2013/00953

(57) ABSTRACT

A sensor unit for detecting a pressure applied to a patient by a medical dressing, the sensor unit comprising: an electronic sensor configured to detect a pressure or force applied to a pressure plate of the electronic sensor; a plurality of lever elements positioned over the pressure plate, wherein each of the plurality of lever elements comprises an abutment region that is moveable towards the pressure plate; and, an exterior plate coupled to the plurality of lever elements such that, in use, a displacement of the exterior plate from an equilibrium position towards the pressure plate causes the abutment region of at least one lever element to move in a direction towards the pressure plate and transfer a force from the exterior plate to the pressure plate.

### BACKGROUND OF THE INVENTION

Venous leg ulcers in particular, are a life-limiting medical condition that responds poorly to current, but long established treatments. This causes long-term distress and possible immobility to the patent, and the long-term nature of the treatment and limited effectiveness makes it an expensive condition to treat.

The key to effective treatment is to bandage (or compress by other means) the leg in such a way that there is a pressure gradient up the leg, starting with a higher pressure (typically 5.2 kPa or 40 mmHg) at the ankle, reducing to 2.6 kPa (or 20 mmHg) at the top of the calf, just below the knee. [Other graduated compression profiles may have an effect.]

Conventional bandaging techniques attempt to achieve such a profile by wrapping a bandage at approximately uniform tension up the leg. The tension may be gauged by noting the amount of stretch for example, in circles pre-imprinted on the bandage. Theoretically, for a uniform tension, the pressure exerted by a bandage on a cylindrical structure (such as approximating a leg) is inversely proportional to the radius of curvature (based on Laplace's Law). This, and the ‘experience’ of the applier of the bandage, is used to produce what is hoped is the correct compression profile. However, typically, the pressures exerted in this way are not as expected (being often higher) and worse, the profile is not uniform. This could make the condition worse as a high spot in the pressure up the leg would tend to inhibit the desired flow of blood and other fluids (such as lymph) up the leg, possibly even causing more serious conditions. In addition, the high pressure and non-uniformity of the pressure gradient may cause unnecessary patient discomfort which results in a low treatment uptake; patients tend to remove the bandage on comfort grounds or even re-fit themselves for comfort, thus completely invalidating the treatment.

Ideally a pressure sensor can be deployed, which is fitted between the leg and bandage (or compression garment, or wrap system) to measure the pressures at points up the leg (typically about 5 or 6). However, it is important that the pressure sensor itself does not cause an erroneous pressure reading by perturbing the pressure where it sits. Typically, inserting an object between the leg and bandage causes a local (possibly dangerous, and definitely a contributor to inaccuracy) elevation in pressure. Attempts to mitigate these difficulties have included making small, thin sensors, but these tend to be expensive, relying on such technologies as fibre gratings and complex interrogation schemes. An added difficulty is that the pressures are quite low, and the sensor needs to be immune to shear and twisting effects.

WO2017/174984 discloses a sensor comprising first and second layers, separated by an elastic spacer. The separation of the first and second layers is determined by capacitive or optical means. This gives an indication of the pressure applied to the top and bottom layers. The sensor is essentially rectangular in cross section.

### SUMMARY OF THE INVENTION

According to a first aspect of the invention, there is provided a sensor unit for detecting a pressure applied to a patient (e.g. to a patient's skin) by a medical (compression) dressing (for example, by a stretch bandage, elasticated hosiery, or a wrap-based system), the sensor unit comprising: an electronic sensor (such as an optical proximity sensor) configured to detect a pressure or force applied to a pressure plate of the electronic sensor; a plurality of lever elements positioned over the pressure plate, wherein each of the plurality of lever elements comprises an abutment region that is moveable towards the pressure plate; and, an exterior plate coupled to the plurality of lever elements such that, in use, a displacement of the exterior plate from an equilibrium position towards the pressure plate causes the abutment region of at least one lever element to move in a direction towards the pressure plate and transfer a force from the exterior plate to the pressure plate.

In the present disclosure, a medical dressing (or more generally “dressing”) should be understood to include any dressing, bandage (such as a stretch bandage), hosiery (such as elasticated hosiery), or a wrap-based system that may be applied to a patient in a relevant medical setting (especially compression dressings). The medical dressing may be in direct contact with a patient's skin, or there may be intermediate layers/dressings between the medical dressing and the patient's skins. The term “bandage” used herein should be understood to refer more generally to such a medical dressing.

When using a sensor to measure the pressure applied by a medical dressing, the sensor needs to be immune to the angle of pressure application as generated by the applied bandage(s). The sensor unit of the first aspect incorporates lever elements positioned between the exterior plate and the pressure plate to ensure that only the normal component of the force exerted by the medical dressing is measured. Two lever elements (or more) are used to transfer the force to the pressure plate. This also means the top (i.e. the exterior plate) does not tip over if the force is applied to one side of the sensor first (as is likely to be the case when applying a bandage).

The exterior plate of the sensor is the part of the sensor unit for receiving a force or pressure from the medical dressing.

Preferably, the abutment region of each respective lever element of the plurality of lever elements is moveable towards the pressure plate by a rotation of the lever element about a pivot joint between the respective lever element and a respective fulcrum positioned adjacent to (i.e. near to but not necessarily directly next to) the electronic sensor. This arrangement allows the relationship between the force/pressure applied to the exterior plate and the force/pressure applied to the pressure plate to be determined using standard classical mechanical equations for torque/moments.

Herein, the term “fulcrum” includes hinges such as living hinges.

Each lever element may be cantilevered, i.e. each lever element may rotate about an end of the of the respective lever element.

Even more preferably, the exterior plate is coupled to each respective lever element at a coupling point positioned between the abutment region and the fulcrum. Coupling the exterior plate to the lever elements in this way means that the force applied to the sensor cell is reduced such that the exterior plate moves less than the pressure plate, which is desirable to reduce geometry changes as more force is applied.

Alternatively, the abutment region of each respective lever element of the plurality of lever elements may be movable towards the pressure plate by a bending of the respective lever element. For example, each of the plurality of lever elements may be deflectable by means of a region of low bending or shear stiffness located along the lever elements.

Optionally, the exterior plate may be coupled to each respective lever of the plurality of lever elements by respective a pin. This arrangement retains the exterior plate to stop it lifting off.

Optionally, the electronic sensor may be a proximity sensor. The electronic sensor may comprise an optical sensor.

Throughout the present disclosure, the pressure/force may be determined indirectly, e.g. by determining a deflection of the pressure plate. In other words the pressure/force may not be explicitly calculated by a sensor, but some other variable which is a proxy for the pressure/force may be determined instead.

Preferably, the pressure plate is a flat spring.

Optionally, the abutment regions of each of the plurality of lever elements may be arranged to directly abut the pressure plate in use. This avoids the need for overlapping lever elements, thereby reducing the heigh of the sensor unit.

Alternatively, the abutment region of a first lever element may be arranged to abut the pressure plate; and, the abutment region of a second lever element may be arranged to abut the first lever element, such that, in use, force is transferred from the abutment region of the second lever element to the pressure plate via the first lever element. This ensures that the force from the exterior plate always acts through a reproducible contact position on the pressure plate, thereby ensuring a consistent pressure/force reading by the electronic sensor.

The sensor unit may optionally comprise at least three lever elements. This increases the stability of the exterior plate, although arrangements are envisaged with only two lever elements.

Optionally, the exterior plate may comprise a cut-out section shaped to receive at least part of each of the plurality of lever elements in use. In other words, the dimensions of the cut-out section may be such that the lever elements do not impinge the exterior plate during operation of the sensor, other than at the linkage between the exterior plate and the lever elements. This allows for the height of the sensor to be reduced.

According to a second aspect of the invention, there is provided a sensor strip comprising an elongate member that is segmented, the segments being defined by continuous longitudinal and transverse grooves, such that adjacent segments are hinged together, wherein the longitudinal grooves define a central column of segments and outer columns of segments, wherein the segments in the outermost columns are tapered outwardly, and wherein a pressure-sensitive sensor comprising the sensor unit of the first aspect is mounted in a plurality of the segments in the central column.

According to a third aspect of the invention, there is provided an electronic pressure sensor for detecting a pressure applied to a patient by a medical dressing, the electronic sensor comprising: a pressure plate arranged to deflect upon application of a force; a rigid base plate opposing the pressure plate and comprising a boss protruding towards the pressure plate; and, a proximity sensor fixedly coupled to the protruding boss and configured to measure a deflection of the pressure plate.

The pressure of the dressing may cause deflections on the order of several microns, and it is important that these deflections are measured accurately. Simply mounting the proximity sensor on a PCB and attaching the PCB to the base means that the proximity sensor can move slightly (e.g. due to flexing or thermal expansion of the PCB), leading to errors in the measurement. The pressure sensor of the third aspect overcomes this problem by fixedly supporting the proximity sensor with a protruding boss, thereby preventing erroneous relative movement between the proximity sensor and the pressure plate.

The pressure sensor may further comprise a support element supporting the pressure plate, wherein the rigid base plate is fixedly coupled to the supporting element. This mitigates the effects of erroneous relative movement between the rigid base plate and the pressure plate.

The pressure sensor of the third aspect may be used as the electronic sensor in the sensor cell of the first or second aspects.

According to fourth aspect of the invention, there is provided a method of correcting temperature variations experienced by a phototransistor in a proximity sensor, comprising: emitting radiation from an LED of the proximity sensor towards an inner surface of a pressure plate of the proximity sensor; obtaining a phototransistor voltage value by measuring a voltage across a phototransistor when radiation reflected from the inner surface of the pressure plate is received at the phototransistor; obtaining an LED voltage value by measuring a voltage across the LED; and, determining a temperature-corrected deflection value based on the phototransistor voltage value and the LED voltage value, wherein the temperature-corrected deflection value is associated with a magnitude of deflection of the pressure plate.

Low-cost phototransistors have a significant temperature drift which adversely affects the accuracy of the sensors. The method of the fourth aspect allows this temperature drift to be corrected without requiring any additional components.

The temperature-corrected deflection value may be a proxy for the magnitude of the deflection rather than the actual magnitude of deflection itself. For example, it may be a temperature-corrected phototransistor voltage value, or it may be a temperature-corrected pressure/force value related to the magnitude of deflection of the pressure plate.

The voltage across the phototransistor is indicative of an uncorrected deflection magnitude of the pressure plate. The (temperature-corrected) magnitude of deflection of the pressure plate represents the magnitude of a force or pressure applied to an external surface of the pressure plate.

The LED voltage value may be determined prior to, at the same time as, or after emitting the radiation and/or obtaining the phototransistor voltage value, although it is preferably obtained at a similar time (i.e. within 5 minutes or more preferably 1 minute of obtaining the phototransistor voltage value) to ensure that the temperature does not change significantly between obtaining the phototransistor voltage value and obtaining the LED voltage value.

One skilled in the art will appreciate that there are numerous ways of determining the temperature-corrected deflection value based on the phototransistor voltage value and the LED voltage value. For example, a temperature correction factor may be determined using the LED voltage value, and temperature-corrected phototransistor voltage value may be determined. Alternatively, an uncorrected pressure value may be determined based on the measured phototransistor voltage, and a temperature correction factor may be applied to the uncorrected pressure value to determine a corrected pressure value.

Optionally, determining the temperature-corrected deflection value may comprise:
determining a temperature correction factor by accessing a lookup table with the LED voltage value, wherein the lookup table is stored in memory on the proximity sensor; and, calculating the temperature-corrected deflection value using the temperature correction factor and the phototransistor voltage value.

Alternatively, determining the temperature-corrected deflection value may comprise: determining a temperature correction value by accessing one or more equation coefficients stored in memory on the proximity sensor; and, calculating the temperature-corrected deflection value using the equation coefficients, the LED voltage value, and the phototransistor voltage value.

Optionally, the method may further comprise generating the lookup table or equation coefficients respectively by: obtaining at least two further LED voltage values, wherein each of the further LED voltage values is obtained at a known pressure and each of the two further LED voltage values is obtained at a different known temperature from the other; and, interpolating between the at least two further LED voltage values. The lookup table/equation coefficients are preferably generated prior to measuring the phototransistor voltage value, e.g. when calibrating the sensor during manufacture.

The method may also comprise determining an ambient temperature based on the measured LED voltage. This may be useful in applications such as determining the temperature of a patient's skin close to the proximity sensor, which can provide an indicator for example, of potential infection (e.g. if the skin temperature is elevated).

According to a fifth aspect of the invention, there is provided a proximity sensor system comprising: a pressure plate; an LED arranged to emit radiation towards an inner surface of the pressure plate; a phototransistor arranged to receive radiation reflected from the inner surface of the pressure plate; and, a processor configured to determine a temperature-corrected deflection magnitude of the pressure plate using the method of the fourth aspect. The processor may be part of the proximity sensor itself, or it may be an external component.

According to a sixth aspect of the invention, there is provided a sensor strip comprising an elongate member that is segmented, the segments being defined by continuous longitudinal and transverse grooves, such that adjacent segments are hinged together, wherein the longitudinal grooves define a central column of segments and outer columns of segments, wherein the segments in the outermost columns are tapered outwardly, and wherein a pressure-sensitive sensor is mounted in a plurality of the segments in the central column; and, a conformable pad shaped to be positioned between a patient-facing surface of the sensor strip and a skin surface of a patient.

Using a conformable pad helps to equalise the pressure under the sensor strip and mitigate the effect of pressure peaks/highlights. The conformable pad may be fixed to the patient-facing surface of the sensor strip (e.g. during manufacture) or it may be a separate component that is applied to the skin surface prior to applying the sensor strip.

The conformable pad may optionally comprise a layer of for example, silicone gel. The layer of gel may be a continuous layer of gel contained within a sealed pouch, or it may comprise a plurality of pouches of gel, wherein each respective pouch of the plurality of pouches of gel is shaped to be positioned between a respective segment of the sensor strip and the skin surface of the patient. Using a gel helps to equalise the pressure locally under each segment of the sensor strip.

Alternatively, the conformable pad may comprise a plurality of pouches of fluid. Fluids generally allow for improved equalisation of the pressure. In the present disclosure, the term fluid should be understood to be a liquid (such as water) or a gas (such as air or nitrogen).

Each respective pouch of the plurality of pouches of fluid or gas is preferably shaped to be positioned between a respective segment of the sensor strip and the skin surface of the patient; pouches of the plurality of pouches of fluid or gas are preferably in fluid communication with each other. For example, the pouches may be in fluid or gas communication by pipes or openings connecting adjacent pouches. Using separate pouches rather than one continuous pouch makes it easier to manipulate the conformable pad to conform to the patient's skin (e.g. around a limb such as a leg).

According to a further aspect of the invention, there is provided a method of determining a pressure under a dressing applied to a patient, comprising: introducing the sensor strip and conformable pad according to the sixth aspect under the dressing with the conformable pressure pad positioned between the sensor strip and a skin surface of the patient; and, recording a pressure at each of the pressure-sensitive sensors.

### DESCRIPTION OF THE FIGURES

Examples of the present invention will now be described in detail, with reference to the accompanying figures, in which:

FIG. 1(a) shows a simplified plan view of an example of an elongate segmented sensor strip;

FIG. 1(b) shows a cross sectional view along the line A-A in FIG. 1(a);

FIG. 1(c) shows cross sectional view along the line B-B in FIG. 1(a) to illustrate the hinging and separation of the segments of the sensor strip in use;

FIG. 2 is a schematic illustration of the elongate sensor strip partially wrapped around the circumference of a section of a leg;

FIG. 3 illustrates the geometry of a segmented sensor strip;

FIG. 4 is for comparative purposes and illustrates alternative sensor geometry;

FIGS. 5(a)-5(d) show plan and cross-sectional views of an example of a sensor strip;

FIGS. 6(a) and 6(b) show side section and plan views of a sensor cell incorporating lever elements;

FIGS. 7(a) and 7(b) show side section and plan views of interior of the sensor cell of FIGS. 6(a) and 6(b);

FIG. 8 shows an exemplary sensor strip; and,

FIGS. 9(a) and 9(b) illustrate fluid pouches for use with the sensor strip of FIG. 8.

### DESCRIPTION OF THE INVENTION

In preferred examples, we measure the displacement of a flexible elongate sensor strip using optical proximity sensors mounted to the strip. Capacitive sensors may also be used. Such a sensor strip is shaped in such a way that it does not significantly alter the pressure exerted by a bandage, in that the sensor strip conforms to the curvature of the leg where it is fitted. In addition, the sensor strip is tapered to avoid any pressure enhancement at the edge of the sensor strip.

In a preferred example, the sensor strip is from 300 to 450 mm long (typically 380 mm long) and 10-15 mm wide. By virtue of the tapering structure and the segmentation to follow the radius of curvature, the sensor strip can be of 2-4 mm thick, which allows cost-effective sensing techniques (the sensor strip will have a short service life, typically days per patient).

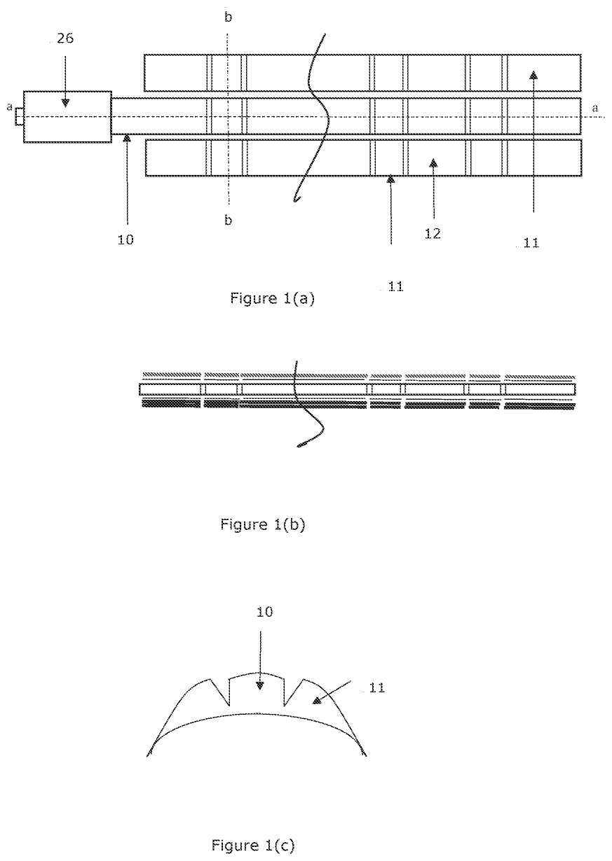

Referring now to the Figures, a sensor strip of the type shown in FIG. 1 can be used by mounting it along a calf with an electrical connector 26 placed just below the knee in typical operation.

The segmentation of the sensor strip allows it to follow the curvature of the leg at any point. The sensor strip comprises a central column 10 and outer columns 11 and each of the segments 12 may have a rigid top and bottom surface.

In preferred examples, the widths of the segments 12 are typically 10 mm, e.g., not more than 20 mm, and as low as 5 mm. The thickness of the sensor strip should be as low as practically possible, but 1-5 mm is a workable range.

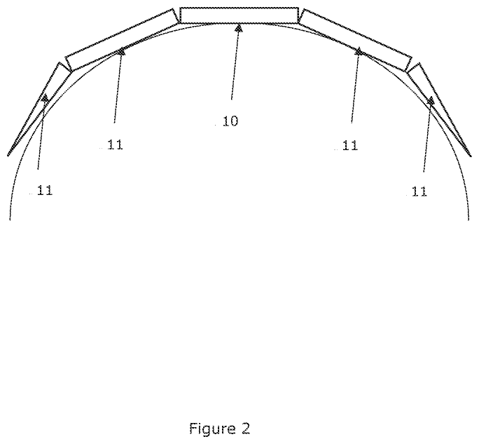

In some examples, there is one outer column 11 either side of the central column 10 as shown in FIG. 1, but it is possible and may enhance performance if there are two outer columns 11 on each side of central column 10—i.e., a total of 5 segments as shown in FIG. 2. Typically, the length of each segment is around 50 mm. This may be dictated by the compliance of the sensor strip and leg geometry and might be as low as 10 mm. 50-60 mm is often the largest practical size.

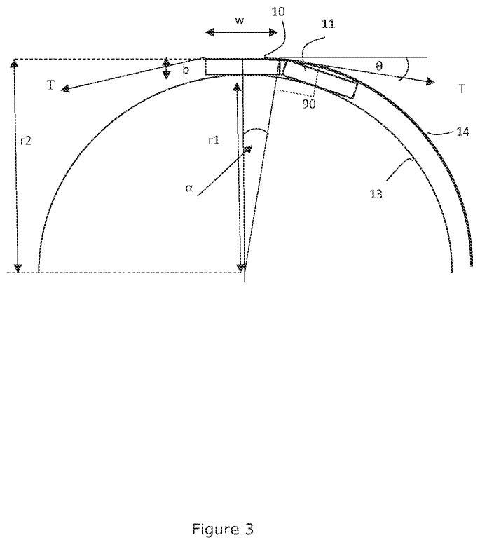

FIG. 3 shows how the sensor strip geometry works. For clarity, only one outer column 11 is shown to the right of the central column 10, as opposed to a sensor strip construction where there is an outer column either side. The drawing shows in addition a mandrel 13 and a bandage 14.

It can be shown that the pressure exerted on the flat upper surface of the sensor's central column 10 will be the same as if the sensor strip was not there (other than a small change of a few percent due to the fact that the sensor thickness of typically 3 mm adds to the effective radius of the leg which might be 35 mm or more), if the bandage leaves the edge of the central column 10 at the correct angle θ, where this angle is the angle of the tangent to the curvature of the bandage shown by the bold curved line. The force exerted on the sensor strip and hence the leg is given by the equation F=Tsinθ. Where F is the downward force and T is the tension in the bandage.

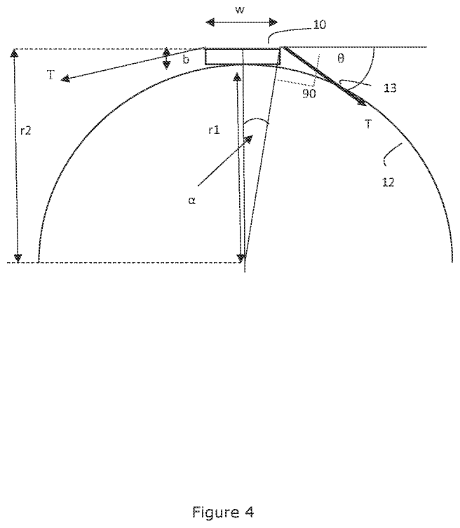

It will be appreciated that if the outer column 11 were not there the sensor strip would read a greatly exaggerated pressure, and this pressure would be exerted locally on the leg, as can be seen from the comparative example in FIG. 4. The pressure could be increased by as much as a factor of 5.

The pivot and segmentation need to ensure the bandage follows to within a certain accuracy the same path as it would if the sensor strip was a continuous (unsegmented) flexible layer, as opposed to rigid segments joined together. It can be shown geometrically and theoretically that as long as the gap between segments is small (<1 mm) and the column width is <15 mm that the error in pressure is <10%. Similar considerations apply to the design of a one-piece segmented structure.

The segmentation allows the use of a relatively rigid material to form the sensor strip of only a few mm in thickness, which enables cost-effective mass manufacturable sensor strips.

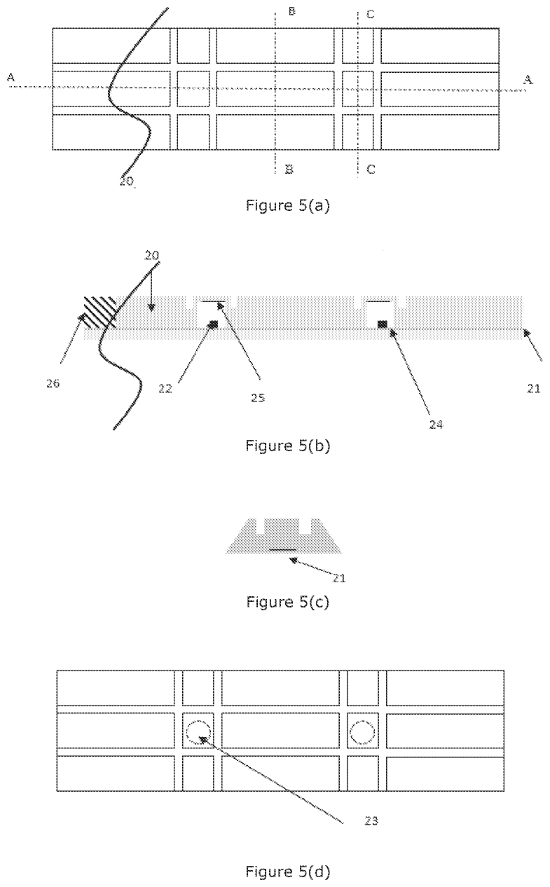

FIG. 5(a) shows a sensor strip formed by mounting a rubber strip 20 on a flexible PCB substrate 21. FIG. 5(b) is a section A-A along FIG. 5(a), which shows in more detail a connector 26, optical proximity sensors 22, each formed in cavity 23, with an (optional) stiffener 24 and reflectors 25. FIG. 5(c) is a section along B-B in FIG. 5(a). The position of each cavity 23 is illustrated in FIG. 5(d).

Preferably, the upper surface of the rubber strip 20 is textured, or a layer of a suitable material is laid on top, in order to enhance contact reproducibility.

In some examples, a sensor strip comprises a one-piece rubber strip or similar structure with a grooved surface to provide the segmentation, below which is attached a flexible PCB to route the signals to the electrical connector and to mount the optical proximity detectors.

FIG. 5(b) shows the flexible PCB 21 running the length of the sensor strip. The sensor cavity 23 is a recess, preferably a circular recess, formed in the rubber strip 20, the internal top surface of which may be coated with suitable reflective material 25, preferably a diffuse Lambertian reflector such as a suitable white paint printed onto the rubber.

In FIG. 5, the top surface of each sensor cavity is shown as square, but circular is an option. A square may be used as it presents a uniform edge for the bandage to land on, given the curved geometry the sensor strip is designed to be used on e.g., a human leg.

The contact area with the bandage should be constant and preferably as close to 100% of the apparent contact area as possible. Black, Closed-cell, Firm Grade Neoprene Foam is a suitable material. The spring constant of the sensor strip and therefore the full-scale excursion of the sensor strip is determined largely by the Youngs Modulus of the rubber foam (HT800), but depends on the area of the recess 23. Optionally, voids could be moulded into the rubber around the sensor cavity to further reduce the area of rubber between the top and bottom of the sensor strip and hence the effective Youngs Modulus of the rubber. There may be an additional rubber layer under the flexible PCB and the stiffeners for encapsulation and to present a uniform surface to the leg.

Another feature is that low-cost phototransistors may be used in the design. However, they have a significant temperature drift which adversely affects the accuracy of the sensors. This can be mitigated by providing an extra proximity detector either next to each sensor or in the centre of the strip, which is set in a cavity that does not respond to pressure. Thus, it will only respond to temperature and this reading can be used to null out any temperature drift. It might be thought that an extra proximity sensor needs to be provided for each sensor but as the sensor will be used under a bandage it is likely it will be at a uniform temperature and so only one compensation device needs to be provided.

The flexible PCB substrate 21 contains the proximity sensors 22 which preferably comprise an LED mounted next to a phototransistor, as is well known to produce a proximity detector. Over a distance of about 1 mm the divergence of the light from the LED ensures that the amount reflected back to the phototransistor varies with distance. The LED and phototransistor are both mounted on the flexible PCB substrate 21 so that the optical axes are parallel and about 1 mm apart, with a barrier to prevent cross talk.

It will be appreciated that other optical configurations are possible, including lenses on the LED or phototransistor to modify the light received vs. distance function. At present, to eliminate errors due to skew or the reflector not being quite parallel to the base of the sensor, we use a Lambertian reflector which has a low angular dependence compared to a specular reflector. A corner cube type reflector could be used instead to avoid skew effects completely. It will also be appreciated that the LED/phototransistor combination could operate at a non-visible wavelength such as infra-red as this allows the phototransistor to incorporate a filter (e.g. an IR pass filter) to reduce ambient light sensitivity, which would otherwise affect the readings. In practice, the sensor is enclosed but the low signal levels mean that even low levels of ambient light can perturb the readings. A standard technique to eliminate this effect further (and also eliminate electrical noise and crosstalk) is used in that the LED is pulsed on and off (at about 1 KHZ), e.g. using a square wave (on and off, 100% modulation). The readings from the phototransistor are taken at LED OFF and LED ON states and this allows subtraction of any offset due to ambient light. The preferred pulsing at 1 KHz also avoids effects due to noise pickup which tends to be at mains frequency of 50 Hz. The readings are preferably only taken from the phototransistor once the reading has settled, which is typically around 100 us after the LED has been turned on. Doing this reduces effects due to ringing or overshoot in the electronics (electrical crosstalk is also more likely on rising or falling edges). Any static leakage (except that from the LED) will be nulled out by the subtraction between the values with LED on and off.

To improve the resolution of the digital to analogue convertors that process the signal from the phototransistors, dither may be used, e.g. by adding a sawtooth modulation to the signal from the phototransistors. Depending on how many samples are taken, using dither allows a 10-bit digital to analogue convertor to achieve a resolution of 12 bits or more. The reading can be updated approximately once per second or as required by the user interface. The higher resolution improves both resolution of the output and accuracy, because readings may be taken at differing temperatures to produce a temperature calibration factor.

As previously discussed, low-cost phototransistors have a significant temperature drift which adversely affects the accuracy of the sensors. For example, the phototransistor current may vary by 0.6% per degree Celsius. A sensor might exhibit 50 mV/mmHg sensitivity, but the actual voltage measured may be 3000 mV. A change of 0.6% would be 18 mV, meaning the temperature drift would lead to an error of 0.36 mmHg per degree Celsius. For a 10° C. temperature shift, this equates to 3.6 mmHg, which is an error of 36% at 10 mmHg.

One way of mitigating this error is by providing the sensor strip with an extra proximity sensor that does not respond to pressure and can be used to null out any temperature drift. However, this requires at least one extra sensor, which increases the cost and complexity of the sensor strip. In addition, the extra sensor may be at a different temperature to the other sensors, in which case it will not provide an accurate correction for the other sensors.

An alternative method of correcting for temperature fluctuations is to use the voltage measured across the LED in each proximity sensor 22. The LED in each proximity sensor 22 is a p-n semiconductor junction and therefore exhibits a measurable temperature characteristic. In general, p-n junctions experience a voltage fluctuation of approximately 2.1 mV/° C., although the exact value depends on several factors in the design and manufacture of the device. However, the temperature variation is generally approximately linear and is reproducible over time, which means the voltage across the LED can be used to infer the temperature of the LED.

In between data collection events (or even whilst data is being collected), the LEDs are fed by a precision current source and the voltage across each LED can be monitored. This allows for real-time measurement of temperature of the LED, which is highly representative of the temperature of the proximity sensor and can be used to correct the sensor readings against temperature. This can be achieved by interpolating between two sensor readings taken at a known pressure and known (and different) temperatures and producing a linear calibration equation, or by generating a look up table so any value can be corrected. This correction method works for all pressure values because the temperature shift is a multiplicative effect on the phototransistor current. As an alternative to a linear interpolation, a polynomial temperature correction curve can be produced to provide even more accurate temperature correction.

The temperature equation (or lookup table) can be stored in memory on the proximity sensor 22 and used by a processor to determine the temperature of the LED and therefore the corresponding temperature correction factor of the phototransistor.

The mechanical components of the sensor do not contribute significantly to the temperature shift, but these contributions would be calibrated out during this process in any case.

In addition to correcting for temperature drift, the temperature signal determined from the LED voltage can be output as an actual ambient temperature value. This may be useful in applications such as determining the temperature of a patient's skin close to the proximity sensor, which can provide an indicator of potential infection (e.g. if the skin temperature is elevated).

The electrical connector of the sensor strip can be coupled to a separate interrogator unit (not shown), typically a small electronics box which could be belt worn by a patient. The interrogator unit houses electronics that are configured to pulse the LEDs and receive the signals from the phototransistors and amplify them using transimpedance amplifiers. The amplifiers are preferably rolled off at about 50 KHz as this allows enough bandwidth whilst keeping noise relatively low. The signal for LED ON and LED OFF is sampled by a microcontroller, but a delay is introduced so the signal can settle before it is sampled. This avoids any effects due to bandwidth limitations affecting the signal. Typically, 100-1000 samples are taken and then averaged to derive a reading.

Other modulation schemes such as a sinusoidal modulation could be used.

The individual sensors may be calibrated hydrostatically or using other methods such as using known weights or applying a force from a rig. A sensor typically will have a transfer function of output voltage vs pressure and this function is linear with both an offset due to manufacturing tolerances and a quadratic term due to the non-linearity of the proximity detector transfer function. The calibration data in the form of the terms a,b,c in the polynomial ax²+bx+c are stored in a non-volatile memory chip (not shown) integrated on the flexible PCB strip. These data are then read by the microcontroller in the interrogation unit and used to calculate an actual pressure reading from the signals from the individual sensors. A temperature correction can also be applied as described above. It should be noted that the individual sensors are not expected to drift excessively during storage and therefore if the voltage at zero pressure is different from the value at calibration (which can also be stored on the memory chip) by too great a margin the sensor will be flagged as faulty. Similarly, in a preferred example the interrogator unit is configured to measure the current drawn by the LEDs and if this is different will be flagged as an error. It would be possible to pinpoint the current change associated with one sensor on the sensor strip failing and to continue using the others (typically 5) on the sensor strip.

Other data are stored on the memory chip for each sensor such as batch number, date of manufacture, date of calibration, number of uses or whether first time use or not (for single use sensors).

In a preferred example, the interrogator unit provided is able to communicate recorded measurements via a USB interface to PC, tablet etc, or to a Bluetooth-enabled device for display on a graphical user interface (GUI). A typical user interface is a histogram type display showing the pressure graded up the leg.

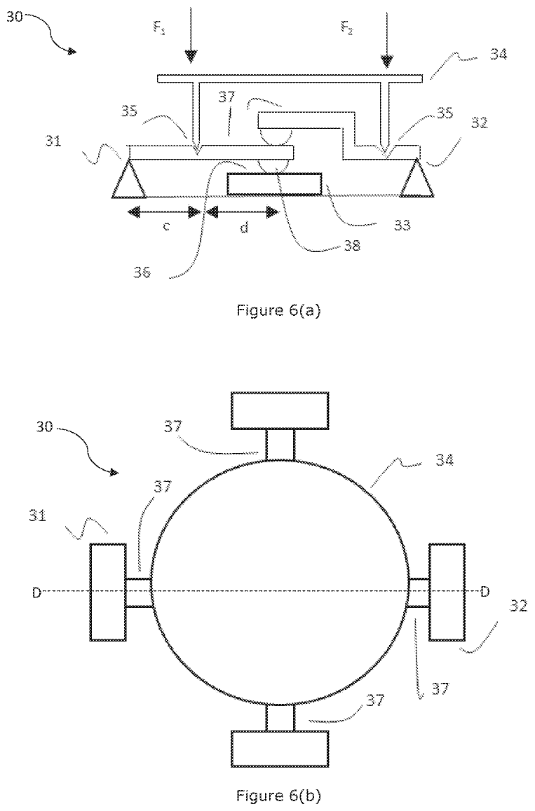

In order to correctly tension the bandage, the sensor strip should be immune to angle of pressure application as generated by the applied bandage. The sensor strip should also be insensitive to any shear forces and other forces and pressures which are not orthogonal to the surface of the leg. The sensor unit 30 shown in FIGS. 6(a) and 6(b) addresses these issues.

A top view of the sensor unit 30 is shown in FIG. 6(b), and a sectional view through the line D-D is shown in FIG. 6(a). For clarity, only two of the lever elements 37 are shown in FIG. 6(a).

The sensor unit 30 comprises an electronic sensor 33, a plurality of lever elements 37 (or levers) pivotable about fulcra 31 and 32, and an exterior plate 34 (also referred to as a top plate) supported by the lever elements 37.

As described in more detail below, the electronic sensor 33 comprises a pressure plate 36 (which could be a flat spring) mounted on a cylindrical washer containing an optical proximity sensor. The pressure plate 36 deflects downwards when a load is applied by the lever elements 37.

The illustrated lever elements 37 are rotatably coupled at one end to fulcra 31 and 32 respectively, with each lever element 37 able to pivot about its respective fulcrum 31, 32.

The exterior plate 34 of the sensor unit 30 is the part that receives a force/pressure from the bandage (i.e. it is in contact with the bandage or has one or more intermediate dressing layers between it and the bandage) and therefore transfers the force applied by the bandage to the pressure plate 36 of the electronic sensor 33 via the lever elements 37. It will be appreciated that the force applied to the electronic sensor 33 will be reduced by the ratio of d/(c+d), and that c and d might be roughly equal. This tends to make the exterior plate 34 move less than the pressure plate 36, which is desirable to reduce geometry changes as more force is applied. In the illustrated example, hemispherical pads 38 are provided on the lever elements 37 to ensure that forces are transferred consistently from one lever element 37 to another, or from a lever element 37 to the pressure plate 36 of the electronic sensor 33, regardless of the relative angles between the lever elements 37 and electronic sensor 33. It should be understood that the hemispherical pads 38 are optional and could be omitted. In addition, while the illustrated lever elements 37 in FIG. 6(a) overlap such that the force from one of the lever elements 37 is transferred to the electronic sensor 33 via the other lever element 37, the lever elements 37 could alternatively each act directly on the pressure plate 36 within a central region of the pressure plate 36 as discussed below.

The illustrated exterior plate 34 bears down on the lever elements 37 at grooves 35 in the lever elements 37. The grooves 35 are wide at the top and become narrower, which permits a degree of relative rotation at the interface between the lever elements 37 and the exterior plate 34, such that the lever elements 37 can rotate as the exterior plate 34 moves downwards. It is also desirable that the exterior plate 34 is retained against the lever elements 37, such that the exterior plate 34 cannot lift off or fall off. A retaining mechanism is not shown in the Figures, but an arrangement is envisaged with pins some way along the lever elements 37 and protruding either side. The exterior plate 34 would have corresponding gripping elements that attach to the pins such that the exterior plate 34 can be retained to the pins by means of an interference/snap fit. It should be understood that the illustrated grooves 35 are optional and the pin arrangement described above could be used without the grooves 35 (i.e. the force from the exterior plate 34 could be transmitted to the lever elements 35 via the pin rather than via grooves 35).

FIG. 6(a) shows a first configuration of the sensor unit 30 in which the lever elements 37 overlap above the centre of the electronic sensor 33. When a force is applied by the exterior plate 34 to the upper lever element 37, it rotates about the pivot point and presses against the lower lever element 37. If a force is also applied by the exterior plate 34 to the lower lever, then the lower lever will also rotate and the force will be transferred onto the pressure plate 36 of the electronic sensor 33. The advantage of this configuration is that the forces are transferred at a reproducible contact position on the electronic sensor 33 relative to the position where the force is applied.

In an alternate configuration (not shown in the Figures), the lever elements 37 can all directly press against the pressure plate 36 of the electronic sensor 33. The lever elements 37 are arranged such that each lever can simultaneously press against the pressure plate 36 as close as possible to the centre, so as to reduce the error in the lever effect from each lever, and make the sensor unit 30 as insensitive as possible to which lever elements 37 are applying a force.

Having the lever elements 37 overlap as illustrated in FIG. 6(a) is desirable to ensure the force always acts through a reproducible contact position on the pressure plate 36, but this arrangement increases the height of the sensor unit 30 which can lead to increased errors in the pressure reading, so it is generally preferred to instead have non-overlapping lever elements 37 that all act directly upon the pressure plate 36.

Referring to the top of FIG. 6(a), it can be seen that if all the force is applied to the left, and assuming there is a retaining means for holding the exterior plate 34 on (e.g. the pin mechanism described above), that the exterior plate 34 would remain flat and not tip over, thus avoiding any unevenness in bandage tension due to the sensor unit 30. When the forces F1 and F2 are roughly equal then the total force is the sum of these two forces. The lever elements 37 will both move and the force will be transferred faithfully to the electronic sensor 33, and reduced by the lever ratio, which is known and fixed. If the forces are not equal, even if one is zero then the same force is still transferred to the sensor. Analytically, the force at the electronic sensor 33 is (F1+F2)*d/(d+c). If a weight is put on top of the exterior plate 34, then the sum of F1 and F2 would equal the force due to the weight, regardless of its position.

If a shear force or torque were to be applied along the exterior plate 34, then the effect would be to try and rotate the exterior plate 34 or displace it sideways. However, in order for the exterior plate 34 to rotate or slide rather than displace vertically, one lever 37 would have to rotate upwards from its fulcrum while the other rotated downwards. A net zero force would be applied to the pressure plate 36 of the electronic sensor 33, either due to the lever elements 37 not contacting the sensor or the lever elements 37 applying equal and opposite forces to the sensor.

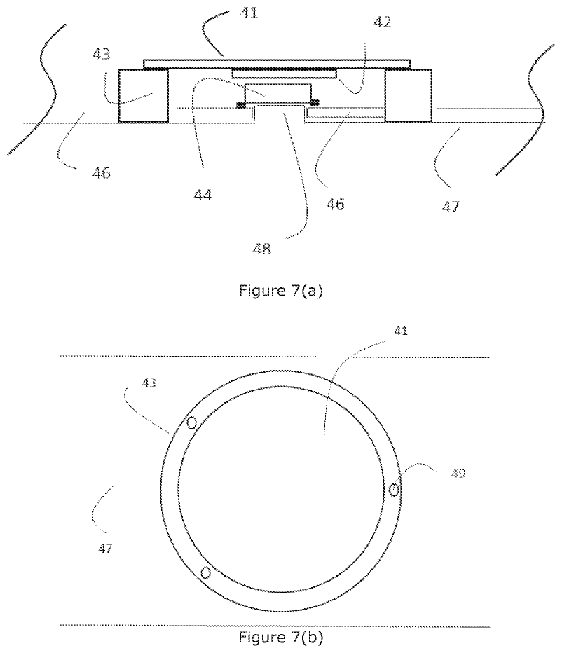

FIG. 7(a) shows the interior of the electronic sensor 33. The pressure plate 41 (e.g. a flat spring) is supported by a support element 43, which may be an annulus or which may have a wider footing than shown. The support element 43 is fixed relative to a base plate 47, for example by having threaded holes in the support element 43 and countersunk screws screwed through the base element 47. The holes and screws together 49 are shown in FIG. 7(b). A proximity sensor 44 is glued or otherwise attached to a boss 48 which protrudes from the base plate 47 (e.g. formed as one piece of moulded plastic, for example). The proximity sensor 44 may be soldered via joints 45 to a flexible PCB 46. A reflector 42 may be attached to the underside of the flat spring 41 and may be any Lambertian reflector. In practice, a plastic part may be attached (glued, snap fit) to the underside of the spring 41 with a reflective material or layer of paint attached to its underside. The distance between the top of the proximity sensor 44 and the top of the reflector 42 is typically 0.5 mm undeflected, although this is dependent on the characteristics of the proximity sensor 44.

It should be understood that the sensor unit 30 and electronic sensor 33 described above can be used in combination with any of the sensor strips shown herein.

While the sensor geometry illustrated in FIGS. 6(a) and 6(b) largely mitigates against errors due to shear forces, inaccuracies can still arise due to pressure peaks/highlights, e.g. at the edges of segments of the pressure sensor. While tapering the sensor strip helps to reduce these effects, the incidence of pressure peaks/highlights can be further mitigated by positioning a layer of gel or fluid between the sensor strip and the patient.

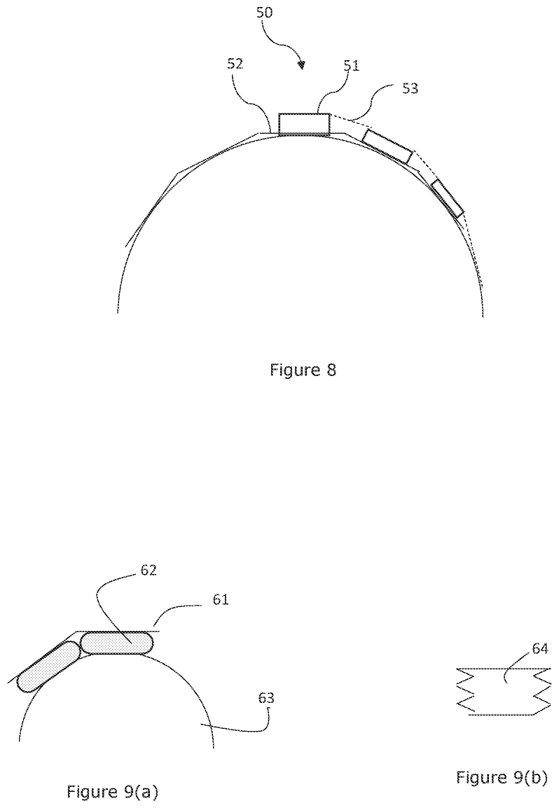

An exemplary sensor strip 50 is shown in FIG. 8, which may be one of the sensor strips described earlier. In this example, pads 51 of each segment of the sensor strip 50 are smaller than rigid base sections 52 on which the pads 51 rest (the pads on the left side of the illustration have been omitted for clarity). It is not necessary for the pads 51 to be as wide as the base sections 52 because the bandage 53 will still follow the correct path when the pads 51 have a smaller area than the bases 52, and a smaller contact area will help with contact area issues. If the pads 51 are too wide compared to the taper angle (i.e. the reduction in height of successive pads) the bandage 53 will not contact properly. For example, the pressure peak if a sensor is 12 mm wide and 4 mm high with no tapering would be four times the anticipated pressure of a bandage on a radius of 35 mm, and four and a half times for a radius of 55 mm.

If the tapering between pads 51 is gradual enough, the angle of the bandage 53 is only increased slightly at each pad 51 and the pressure elevation is within acceptable limits (e.g. a tapering width of around 50 mm will result in a pressure increase of around 10%). This pressure elevation can be further mitigated by positioning a conformable pad in under the sensor strip in the form of a layer of gel, which could either be a continuous layer or segmented.

When using a continuous layer of gel, the gel is preferably contained within a sealed pouch (e.g. a rubber bag) with thin walls (i.e. of negligible thickness compared to/at least one order of magnitude thinner than the thickness of the gel layer) that are have a compliance large enough so as exert non-negligible pressure when bent round the limb. This can be achieved using a silicone gel sandwiched between two layers of cured silicone rubber, although other materials could also be used.

The gel will act to remove the pressure highlights, although it will not completely equalize the pressure everywhere as it is a gel not a fluid. However, when a bandage or dressing is applied over the sensor strip 50, the segments will arrange themselves to give a uniform pressure without highlights even at the edge. This can be visualized as the outer segment, subjected to a higher force at the outer edge, will tend to tip, which will result in an equilibrium when the angle of the bandage is reduced to the point where the force on the outer segment balances the restoring forces from the gel. There will be a pressure elevation under the segments, but this will be uniform and of the order of 10% if the tapering is sufficient.

Using as segmented pad with individual gel pouches under each flap acts to remove the unevenness due to the flat segments pushing on a curved surface, and this arrangement also removes the requirement of the rubber container to be so elastic.

Instead of using a gel, a conformable pad containing a fluid (i.e. a liquid or gas) could be used to mitigate the pressure highlights and equalise the pressure beneath the sensor strip. In this arrangement, each segment/flap 61 of the sensor strip is provided with a separate fluid-filled pouch 62 (alternatively referred to as a bladder or sac) as shown in FIG. 9(a).

To allow for the pressure to be equalised, the fluid-filled pouches 62 are all in fluid communication with each other (e.g. interconnected by small pipes or openings between adjacent bladders 62). In this case, the tendency of a vessel full of air to adopt a spherical shape can be controlled compared to using a single continuous bladder or pouch, and each bladder 62 doesn't have to stretch excessively as it is bent or deformed round the mandrel or limb 63, thus allowing a completely even pressure distribution to be applied to the limb 63. The edge effect will be eliminated by the tilting of the outer taper as described earlier, and all segments will adopt the position required (which will tend to be circular).

The pouches 62 may be pouches of fluid as shown in FIG. 9(a), or they may alternatively be interconnected bellows type structures 64 as shown in FIG. 9(b).

In the above arrangements, the thickness of the fluid or gel layer is preferably chosen to be thick enough to accommodate the tipping of the outer flaps, which will generally be 1-3 mm.

The invention claimed is:

1. A sensor unit for detecting a pressure applied to a patient by a medical dressing, the sensor unit comprising:
an electronic sensor configured to detect a pressure or force applied to a pressure plate of the electronic sensor;
a plurality of lever elements positioned over the pressure plate, wherein each of the plurality of lever elements comprises an abutment region that is moveable towards the pressure plate; and,
an exterior plate coupled to the plurality of lever elements such that, in use, a displacement of the exterior plate from an equilibrium position towards the pressure plate causes the abutment region of at least one lever element to move in a direction towards the pressure plate and transfer a force from the exterior plate to the pressure plate.

2. The sensor unit of claim 1, wherein the abutment region of each respective lever element of the plurality of lever elements is moveable towards the pressure plate by means of a pivot joint joining the respective lever element to a fulcrum positioned adjacent to the electronic sensor.

3. The sensor unit of claim 2, wherein the exterior plate is coupled to each respective lever element at a coupling point positioned between the abutment region and the fulcrum.

4. The sensor unit of claim 1, wherein the abutment region of each respective lever element of the plurality of lever elements is moveable towards the pressure plate by a bending of the respective lever element.

5. The sensor unit of claim 1, wherein the exterior plate is coupled to each respective lever of the plurality of lever elements by a respective a pin.

6. The sensor unit of claim 1, wherein the electronic sensor is a proximity sensor.

7. The sensor unit of claim 1, wherein the electronic sensor comprises an optical sensor.

8. The sensor unit of claim 1, wherein the pressure plate is a flat spring.

9. The sensor unit of claim 1, wherein the abutment regions of each of the plurality of lever elements are arranged to directly abut the pressure plate.

10. The sensor unit of claim 1, wherein:
the abutment region of a first lever element is arranged to abut the pressure plate; and,
an abutment region of a second lever element is arranged to abut the first lever element, such that, in use, force is transferred from the abutment region of the second lever element to the pressure plate via the first lever element.

11. The sensor unit of claim 1, comprising at least three lever elements.

12. The sensor unit of claim 1, wherein the exterior plate comprises a cut-out section shaped to receive at least part of each of the plurality of lever elements in use.

13. An electronic pressure sensor for detecting a pressure applied to a patient by a medical dressing, the electronic sensor comprising:
a pressure plate arranged to deflect upon application of a force;
a rigid base plate opposing the pressure plate and comprising a boss protruding towards the pressure plate; and,
a proximity sensor fixedly coupled to the protruding boss and configured to measure a deflection of the pressure plate.

14. The electronic pressure sensor of claim 13, further comprising a support element supporting the pressure plate, wherein the rigid base plate is fixedly coupled to the support element.

15.-19. (canceled)

20. A proximity sensor system comprising:
a pressure plate;
an LED arranged to emit radiation towards an inner surface of the pressure plate;
a phototransistor arranged to receive radiation reflected from the inner surface of the pressure plate; and,
a processor configured to determine a temperature-corrected deflection magnitude of the pressure plate using the method of:
emitting radiation from an LED of the proximity sensor towards an inner surface of a pressure plate of the proximity sensor;
obtaining a phototransistor voltage value by measuring a voltage across a phototransistor when radiation reflected from the inner surface of the pressure plate is received at the phototransistor;
obtaining an LED voltage value by measuring a voltage across the LED; and,
determining a temperature-corrected deflection value based on the phototransistor voltage value and the LED voltage value, wherein the temperature-corrected deflection value is associated with a magnitude of deflection of the pressure plate.

21.-28. (canceled)
